(19) United States
(12) Patent Application Publication
(10) Pub. No.: US 2005/0148964 A1
(43) Pub. Date: Jul. 7, 2005

(54) Absorbent structure having profiled stabilization

(76) Inventors: Leon Eugene Chambers JR., Cumming, GA (US); Stephen Foster Borengasser, Neenah, WI (US)

Correspondence Address: Melanie I. Rauch;Pauley Petersen & Erickson, Suite 365, 2800 West Higgings Road, Hoffman Estates, IL, 60195, US

(21) Appl. No.: 10/748,000

(22) Filed: Dec. 29, 2003

(51) Int. Cl. A61F 13/15; A61F 13/20

(52) U.S. Cl. 604/367

(57) ABSTRACT

A conformable, absorbent structure having profiled stabilization and, consequently, decreased stiffness, includes an absorbent material at least partially stabilized with a binder material and located in a central longitudinal region and in two distal longitudinal regions located along opposite sides of the central longitudinal region. The absorbent material is more stabilized in the central longitudinal region than in either of the two distal longitudinal regions. The absorbent structure can be included in a number of absorbent articles, including diapers, training pants, swimwear, absorbent underpants, adult incontinence products, and feminine hygiene products, for example. The invention also includes a method of forming an absorbent structure having profiled stabilization.

### BACKGROUND OF THE INVENTION

This invention is directed to a partially stabilized absorbent structure having profiled stabilization, resulting in decreased overall stiffness of the structure. This invention is also directed to methods of forming such absorbent structures.

Absorbent structures are typically present within absorbent articles to provide absorbent capacity. In order to provide structural integrity when the absorbent structure becomes wet, the absorbent structure is typically stabilized. Stabilization of absorbent structures in personal care products has also been demonstrated to yield improved fluid handling properties through enhanced fluid intake and dimensional stabilization of the absorbent core during use.

Recent developments in the use of stabilized airformed composites have utilized bicomponent staple fibers as the binder of choice to achieve a stabilized absorbent matrix. A typical stabilized airformed absorbent structure contains a staple bicomponent binder fiber in a ratio of approximately 3% to 10% by weight of the absorbent material. In such an airformed composite, these fibers may be homogeneously mixed with pulp and superabsorbent in an airforming process. Once formed, the absorbent is heated which evenly melts the sheath of the bicomponent binder fibers allowing them to bond to themselves and to the other components homogeneously throughout the composite. As a result, tensile strength, permeability, and stiffness are equivalent in all planes (cross direction, machine direction, and z-direction) of the absorbent structure.

Everything else being equal, such as density, stabilized absorbents are stiffer than unstabilized composites. As a result of the matrix stabilization, the magnitude of associated stiffness depends on several factors such as the ratio by weight of binder fiber, the degree to which binder fiber is melted, and the type of binder fiber utilized, taking into account the binder fiber's melting point, affinity for the other components in the matrix, and other material properties. For example, in the case of higher binder fiber ratio, a composite containing 6% binder would contain a higher bond point density than a material containing 3% binder. As bond point density increases, the stiffniess also increases. Also, as bond point quality (magnitude and efficiency) increases, greater stiffness results.

The primary benefits of stabilizing an absorbent structure for a personal care product execution are permeability and integrity. Both of these properties are directly proportional to the amount and quality of the binder fiber bonds in the absorbent structure. In a typical case, the integrity and permeability of the absorbent structure are high throughout all planes of the absorbent structure. If stabilization is uniform throughout the structure, stiffness will also be equivalent throughout.

One drawback of stabilized absorbent structures is the increased cross-directional (CD) stiffness associated with a stabilized matrix. This increased stiffness results in consumer discomfort during use in a diaper, incontinence product, feminine care product, or other personal care product.

There is thus a need or desire for an absorbent structure that provides the permeability and integrity of a stabilized absorbent structure with decreased stiffness for enhanced comfort.

### SUMMARY OF THE INVENTION

This invention is directed to conformable absorbent structures having profiled stabilization, absorbent articles including such absorbent structures, and methods of forming such absorbent structures.

The absorbent structures include a central longitudinal region and two distal longitudinal regions located along opposite sides of the central longitudinal region. For purposes of the invention, the term “longitudinal” refers to the lengthwise direction of the absorbent structure, generally extending from a front side or portion of a wearer to a back side or portion of a wearer during use. A target zone of the structure is located in the central longitudinal region. The target zone is the part of the absorbent structure that is insulted by a wearer, such as where the urine stream is injected in a diaper. An absorbent material, or combination of absorbent materials, is located in the central longitudinal region as well as in each of the distal longitudinal regions. The absorbent material is at least partially stabilized with a binder material, thereby creating profiled stabilization in a cross-direction (CD) of the structure, with increased stabilization in the central longitudinal region compared to the two distal longitudinal regions. The stabilization profile may be gradual, as opposed to distinct central and distal longitudinal regions. This profiled stabilization allows for the control of stiffness and certain fluid handling characteristics, such as capillarity.

Permeability is most important in the target zone because fluid must be absorbed quickly or the chances of leakage will be high. Because high permeability and integrity are most necessary in the center of the absorbent structure, stabilization (and stiffness) is also most necessary in this area. Thus, stabilization (and stiffness) can be decreased in the distal longitudinal regions of the absorbent structure. In addition to the increased comfort of the product resulting from decreased stiffness, a fluid functionality improvement is another beneficial result. The higher capillarity of the unstabilized material in the distal longitudinal regions draws fluid away from the central longitudinal region of the absorbent structure. This capillary action allows for improved overall efficiency of the absorbent structure.

The profiled stabilization of the absorbent structure can be accomplished using various approaches. In certain embodiments, the central longitudinal region may include a higher concentration of bond points than in either of the distal longitudinal regions. In other embodiments, bond point concentration across the central longitudinal region and the distal longitudinal regions may be essentially uniform, with lower quality (or weaker) bond points in the distal longitudinal regions.

In one embodiment, different binder materials may be used in different regions of the absorbent structure. For example, a binder that melts at a higher temperature can be utilized in the distal longitudinal regions of the absorbent structure, and a binder that melts at a lower temperature can be utilized in the central longitudinal region of the absorbent structure.

In another approach, the amount of binder can be varied. An absorbent structure that contains little or no binder material in the distal longitudinal regions, and a greater binder-material-to-absorbent-material ratio in the central longitudinal region, such as about 6%, will be less stiff than one that has a binder-material-to-absorbent-material ratio of about 6% throughout. The amount of absorbent material may be uniform throughout the absorbent structure, or may vary as well between the central longitudinal region and the distal longitudinal regions. In yet another approach, the amount of binder activation can be varied in the absorbent structure. More energy can be added to the central longitudinal region of the structure, allowing for fewer and/or weaker bond points in the distal longitudinal regions. Alternatively, or in addition to some of these approaches, the bond points in the distal longitudinal regions can be broken, such as by pleating, subsequent to activating the binder material. In several of these approaches, the absorbent material may be homogeneously mixed with the binder material throughout each of the regions, yet the resulting structure may have a greater concentration of bond points in the central longitudinal region than in either of the distal longitudinal regions. Alternatively, the resulting structure may have essentially the same concentration of bond points throughout the central longitudinal region and the distal longitudinal regions, but the bond points in the distal longitudinal regions are less stabilized than the bond points in the central longitudinal region.

Additionally, the amount of stabilization may also vary in a z-direction of the absorbent structure, thereby creating a z-direction stabilization profile. In this embodiment, a body side surface of the absorbent structure may have greater stabilization with less stabilization on an outer surface of the absorbent structure.

The binder material can be binder fibers, such as bicomponent staple fibers, non-adhesive or adhesive extruded fibers such as meltsprayed or meltblown fibers, or sprayed liquid binder, or any combination of these binder materials. The absorbent material may be an airformed composite, and may include fiberized pulp fibers and, optionally, superabsorbent material. The basis weight of the absorbent structure may be essentially the same in the central region as in each of the two distal longitudinal regions, or the central region may have a higher basis weight due to additional binder material.

In yet another embodiment, the absorbent structure may include a non-stabilized layer of absorbent material adjacent to a surface of the at least partially stabilized absorbent material. The non-stabilized layer may have a transverse width about equal to a transverse width of the at least partially stabilized absorbent material, or may extend past the distal longitudinal regions of the at least partially stabilized absorbent material.

The absorbent structures of the invention, including the absorbent structures resulting from the methods of the invention, may be incorporated into any suitable absorbent article. More particularly, the absorbent structure may be positioned between an outer cover and a body side liner of an absorbent article. Examples of such absorbent articles include, but are not limited to, infant diapers, child training pants, swimwear, absorbent underpants, adult incontinence products, feminine care products, and the like, as well as medical absorbent articles such as medical absorbent garments, bandages, masks, wound dressings, underpads, wipes, and the like.

Matrix stabilization (incidence and/or quality of bond points) decreases from the center of the absorbent structure towards the CD edge. This stabilization profile results in a reduced stiffness in the CD of the material such that the distal longitudinal regions, located closest to the inside thighs of the wearer, yield more easily and provide increased comfort during use. In addition, the portions of the absorbent structure that are less stabilized have better capillarity such that fluid is drawn away from the target zone towards the CD edges of the absorbent structure.

With the foregoing in mind, it is a feature and advantage of the invention to provide an absorbent structure having profiled stabilization, and methods of forming such absorbent structures.

### BRIEF DESCRIPTION OF THE DRAWINGS

FIG. 1 is a plan view of an absorbent structure.

FIGS. 2-6 are expanded cross-sectional views taken along line 2-2 in FIG. 1, each illustrating different embodiments of profiled stabilization of the absorbent structure.

FIG. 7 is a plan view of another embodiment of an absorbent structure.

FIG. 8 is an expanded cross-sectional view taken along line 8-8 in FIG. 7.

FIG. 9 is a perspective view of an absorbent article into which an absorbent structure may be incorporated.

FIG. 10 is a plan view of an absorbent article with an absorbent structure incorporated therein, with the absorbent article in a stretched flat state and showing the body-contacting surface of the absorbent article.

FIG. 11 is a perspective view of a cylindrical compression testing apparatus.

FIG. 12 is a top view of the cylindrical compression testing apparatus of FIG. 11 with a test sample in place.

FIG. 13 is a perspective view of the cylindrical compression testing apparatus of FIG. 11 with a test sample in place during testing.

### DEFINITIONS

Within the context of this specification, each term or phrase below will include the following meaning or meanings.

“Absorbent article” includes personal care absorbent articles such as diapers, training pants, swimwear, absorbent underpants, adult incontinence products, feminine care products, and the like, as well as medical absorbent articles such as medical absorbent garments, bandages, masks, wound dressings, underpads, wipes, and the like.

“Airformed” refers to a mat including cellulosic fibers such as those from fluff pulp that have been separated, such as by a hammermilling process, and then deposited on a porous surface. Other staple fibers or particulates can be included in the mat, such as polyethylene terephthalate for added resiliency, or superabsorbent particles for added absorbency. Airfelt, airlaid, and conform materials are considered within the scope of the term “airformed” as used herein.

“Airlaid” refers to a fibrous structure formed primarily by a process involving deposition of air-entrained fibers onto a mat, typically with binder fibers present, and typically followed by densification and thermal bonding.

“Binder” refers to a material having the capacity to bind itself to another material or to itself, or to bind two or more materials together and/or to form a matrix of intertwined fibers which provide stability to a fibrous and/or particulate structure, under suitable conditions.

“Bond point” refers to an area at which a binder material adheres to another material or to itself, such as an absorbent fiber, group of absorbent fibers, superabsorbent particles, or another binder material.

“Conform” refers to a type of airformed material produced by combining air-entrained dry, dispersed cellulosic fibers with meltblown synthetic polymer fibers while the polymer fibers are still tacky.

“Gradual stabilization profile” refers to a structure having regions of greater and lesser (or no) stabilization, wherein the transition between such regions is not an abrupt transition but instead includes a gradual increase/decrease in the amount of stabilization between such regions.

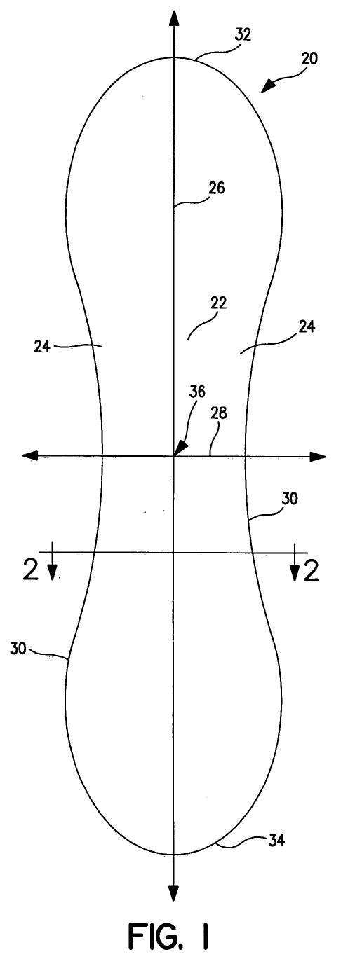

“Longitudinal” and “transverse” have their customary meaning, as indicated by the longitudinal and transverse axes depicted in FIG. 1. The longitudinal axis lies in the plane of the article and is generally parallel to a vertical plane that bisects a standing wearer into left and right body halves when the article is worn. The transverse axis lies in the plane of the article generally perpendicular to the longitudinal axis. The absorbent structure as illustrated is longer in the longitudinal direction than in the transverse direction.

“Meltblown fiber” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., incorporated herein in its entirety by reference. Meltblown fibers are microfibers which may be continuous or discontinuous, are generally smaller than about 10 microns in diameter, and are generally entangling and/or self-bonding when deposited onto a collecting surface. Meltblown fibers may also be macrofibers, namely 10-100 micron fibers or fiber bundles.

“Meltspray” refers to a process used for forming fibers by extruding molten thermoplastic material through a plurality of fine die capillaries as molten threads or filaments into converging, diverging, and tangential, high-velocity heated gas (e.g., air) streams which attenuate the filaments of molten thermoplastic material through the use of one or more high throughput dies which utilize primary and optionally secondary attenuation air to draw the molten polymer into fibers. Such a process is disclosed, for example, in U.S. Pat. No. 5,227,107 to Dickenson et al., incorporated herein in its entirety by reference. Conventional meltspray equipment has the ability to cycle on and off the production of fibers due to the use of a pneumatic needle valve which is located within the fiber channel.

“Pulp fibers” refers to a material made up of cellulose fibers. The material is typically lightweight and has absorbent properties.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat, No. 4,340,563 to Appel et al., and U.S. Pat. No. 3,692,618 to Dorschner et al., U.S. Pat. No. 3,802,817 to Matsuki et al., U.S. Pat. Nos. 3,338,992and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average diameters larger than about 15 microns, more particularly, between about 18 and 40 microns.

“Stabilized” refers to material having a sufficient concentration of bond points to help maintain the structure of the material under force. “Non-stabilized” refers to a material having few or no bond points such that the material can be easily deformed under force.

“Superabsorbent” refers to a water-swellable, water-insoluble organic or inorganic material capable, under the most favorable conditions, of absorbing at least about 15 times its weight and, more desirably, at least about 25 times its weight in an aqueous solution containing 0.9 weight percent sodium chloride. The superabsorbent materials can be natural, synthetic, and modified natural polymers and materials. In addition, the superabsorbent materials can be inorganic materials, such as silica gels, or organic compounds such as cross-linked polymers. The superabsorbent material may be biodegradable or non-biodegradable. The superabsorbent materials can include particles, fibers, tows, flakes, films, foams, and the like. A material is “absorbent” if it absorbs at least five times its weight of the aqueous solution under these conditions.

“z-direction” refers to a direction perpendicular to a plane in which the transverse and longitudinal directions are defined.

These terms may be defined with additional language in the remaining portions of the specification.

### DESCRIPTION OF PREFERRED EMBODIMENTS

In accordance with the invention, absorbent structures having profiled stabilization can be achieved using the methods described herein. These absorbent structures have decreased stiffness and improved fluid handling properties compared to homogeneously stabilized absorbent structures.

Referring to FIG. 1, an absorbent structure 20 is illustrated. The absorbent structure 20 has three longitudinal regions, namely a central longitudinal region 22 and two distal longitudinal regions 24 located along opposite sides of the central longitudinal region 22. The absorbent structure 20 includes an absorbent material at least partially stabilized with a binder material. More particularly, the absorbent material is more stabilized in the central longitudinal region 22 than in either of the two distal longitudinal regions 24.

The terms “central longitudinal region” and “distal longitudinal region” are used to provide a conceptual understanding of the stabilization profile of the absorbent structure 20. These regions are not necessarily separate and distinct locations. More particularly, the central longitudinal region 22 is established along a longitudinal centerline 26 of the absorbent structure 20 and emanates in a transverse direction from the longitudinal centerline 26. The distal longitudinal regions 24 are established along distal edges 30 of the absorbent structure and emanate in a transverse direction toward the central longitudinal region 22. There is not necessarily a line of demarcation separating the central longitudinal region 22 from the distal longitudinal regions 24, and, in fact, the regions may overlap one another such that a gradual stabilization profile is established in a transverse direction of the absorbent structure 20. For the purposes of the invention, when referring to quantitative properties of each of these regions, the measurements of the central longitudinal region 22 are taken along the longitudinal centerline 26, suitably at the intersection with a transverse centerline 28; similarly, the measurements of the distal longitudinal regions 24 are taken along the distal edges 30, suitably at the intersection with the transverse centerline 28. For reference, the longitudinal centerline 26 and the transverse centerline 28 depicting the orientation of the longitudinal axis and the transverse axis, respectively, of the absorbent structure 20 are illustrated in FIG. 1. In typical manufacturing processes, the longitudinal direction is aligned in the machine direction (MD), and the transverse direction is aligned in the cross direction (CD).

The central longitudinal region 22 need not extend a full length of the absorbent structure 20, but may suitably include about 25% or more of a width of the absorbent structure 20 measured along the transverse centerline 28, and may suitably include about 25% or more of the length of the absorbent structure 20 measured along the longitudinal centerline 26, as illustrated in FIG. 1. For example, an absorbent structure 20 having a length of 12 inches (30.5 cm) and a crotch width of 4 inches (10.2 cm) may have a central longitudinal region 22 of about 3 inches (7.6 cm) in length and about 1 inch (2.5 cm) in width. Although illustrated as an hourglass shape, the absorbent structure 20 may alternatively be rectangular, oblong, irregular, or any other suitable shape. Similarly, the central longitudinal region 22 and the distal longitudinal regions 24 may either follow the overall contour of the absorbent structure 20 or may form any other suitable shape within the confines of the absorbent structure 20.

The central longitudinal region 22 may include a higher concentration of bond points than in either of the two distal longitudinal regions 24, thus creating profiled stabilization in the CD of the absorbent structure 20. Alternatively, the central longitudinal region 22 may include an essentially uniform bond point concentration across the central longitudinal region 22 and the distal longitudinal regions 24 with weaker bond points in the distal longitudinal regions, thus also creating profiled stabilization in the CD of the absorbent structure 20.

A target zone 36 of the absorbent structure 20 is located in the central longitudinal region 22. The target zone 36 is the part of the absorbent structure 20 that is insulted by a wearer, such as where the urine stream is injected in a diaper. The greater stabilization of the central longitudinal region 22, in comparison with the distal longitudinal regions 24, allows for optimum intake properties, such as integrity and permeability, where the functionality is needed most. Permeability and integrity are most important in the target zone 36 because fluid must be absorbed quickly or the chances of leakage will be high.

Matrix stabilization (incidence and/or quality of bond points) is decreased in the distal longitudinal regions 24 compared to the central longitudinal region 22. Thus, the distal longitudinal regions 24 are less stiff than the central longitudinal region 22. This profiled stabilization results in an overall reduced stiffness in the transverse direction, or CD, of the absorbent structure 20. Consequently, when the absorbent structure 20 is positioned between a wearer's thighs, as would be the case in a number of personal care absorbent articles containing the absorbent structure 20, the absorbent structure 20 yields more easily and provides increased comfort during use compared to a fully stabilized absorbent structure. A demonstration of the reduced stiffness is provided in the Example below. In addition, the less-stabilized distal longitudinal regions 24 have better capillarity in a wetted state than the stabilized central longitudinal region 22 such that after the target zone 36 is saturated, any additional fluid is drawn away from the target zone 36 towards the CD edges of the absorbent structure 20. More particularly, the absorbent structure 20 may be considered an “active” absorbent structure in that the absorbent structure 20 responds to a user's inputs. That is, when the absorbent structure 20 becomes wet, the edges 30 or the distal longitudinal regions 24 collapse, thereby creating increased capillarity. The result of this profiled stabilization is a product having improved overall efficiency that benefits from stabilization (increased permeability and integrity) in the target zone 36, but suffers minimally from the negative tactile properties (stiffness) associated with a stabilized absorbent.

Another feature of the invention is that the greater stabilization in the central longitudinal region 22 creates a stabilized channel in the MD that keeps fluid moving in the MD, either towards a front longitudinal edge 32 or towards a back longitudinal edge 34 of the absorbent structure, during fluid introduction. Thus, if incoming fluid cannot move to the distal longitudinal regions 24, such as when the distal longitudinal regions 24 are saturated or capillarity is slow, the fluid can move either forward or backwards in the central longitudinal region 22. Consequently, the target zone 36 remains capable of handling additional incoming fluid.

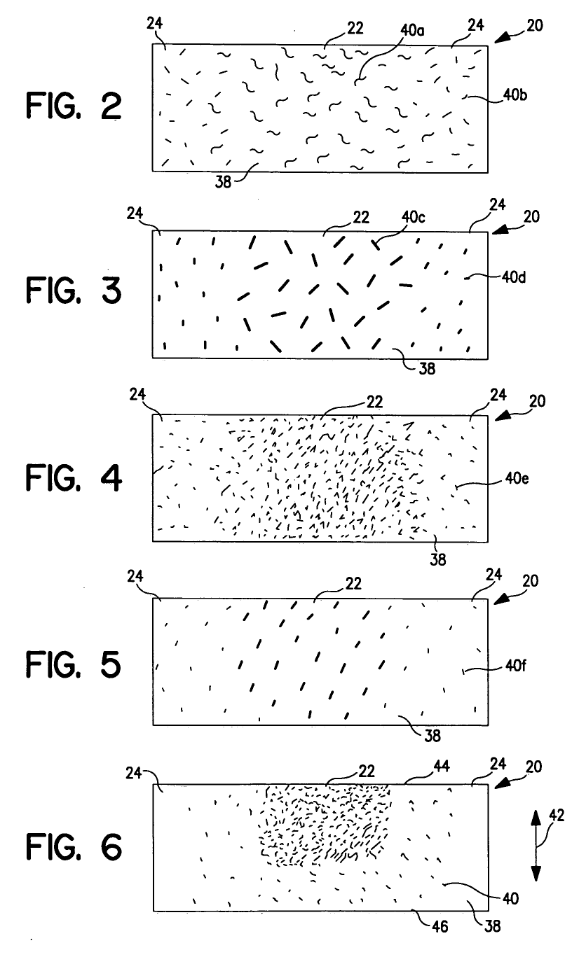

FIGS. 2-6 illustrate cross-sectional views of various embodiments of the absorbent structure 20, taken along line 2-2 of FIG. 1. As mentioned, the absorbent structure 20 includes an absorbent material 38 that is at least partially stabilized with a binder material 40. Although the concentration of bond points may be greater in the central longitudinal region 22 than in either of the distal longitudinal regions 24, as shown in FIG. 4, the concentration, or amount by weight, of absorbent material 38 may be essentially the same, or different, in the central longitudinal region 22 as in each of the distal longitudinal regions 24. As shown in FIGS. 2 and 3, the concentration of bond points may be essentially uniform throughout the central longitudinal region 22 and the distal longitudinal regions 24, but the bond points may be stronger in the central longitudinal region 22 than in either of the distal longitudinal regions 24.

The stabilized, or partially-stabilized, absorbent structure 20 can be formed by forming the absorbent material 38 into a single absorbent structure 20 including the central longitudinal region 22 and two distal longitudinal regions 24 located along opposite sides of the central longitudinal region 22. The absorbent structure 20 can be stabilized with the binder material 40 in such a way that the central longitudinal region 22 is more stabilized than either of the two longitudinal regions 24. Several approaches for creating the stabilization profile are described in greater detail below.

One approach for forming the absorbent structure 20 having a stabilization profile is to vary the type of binder material 40, as illustrated in FIG. 2. For example, a large concentration of continuous fiber binder material 40a can be mixed with the absorbent material 38 in the central longitudinal region 22, and primarily discontinuous fiber binder material 40b can be mixed with the absorbent material 38 in the distal longitudinal regions 24. In this embodiment, the amount of binder material 40 by weight may be essentially the same in the central longitudinal region 22 as in the distal longitudinal regions 24, or the amount of binder material 40 may vary among the regions. Additionally, the number of binder fibers in each region may be essentially the same or different. In any of these variations, the resulting absorbent structure 20 may have increased stabilization in the central longitudinal region 22 due to differences between the binder materials 40a and 40b.

Another example of using different types of binder material 40 in an absorbent structure 20 to achieve a stabilization profile is illustrated in FIG. 3. In this embodiment, one type of binder material 40c having a lower melting point can be mixed with the absorbent material 38 in the central longitudinal region 22, and another type of binder material 40d having a higher melting point can be mixed with the absorbent material 38 in the distal longitudinal regions 24. Once activated, namely through the application of heat, the binder material 40c having the lower melting point will melt sooner and will form a higher concentration of bond points at a lower temperature than the binder material 40d having the higher melting point. In this embodiment, the amount of binder material 40c/40d may be the same in the central longitudinal region 22 as in the distal longitudinal regions 24. For example, the binder material 40c/40d may be uniformly distributed in an amount between about 2% and about 20% by weight of the absorbent structure 20, but the difference in melting points or other physical characteristics will result in greater stabilization in the central longitudinal region 22 than in either of the distal longitudinal regions 24.

Another approach for forming the absorbent structure 20 having a stabilization profile is to vary the amount of binder material 40 across the CD or transverse direction 28 of the absorbent structure 20, as shown in FIG. 4. More particularly, a greater binder-material-to-absorbent-material ratio can be present in the central longitudinal region 22 compared to the binder-material-to-absorbent-material ratio in the distal longitudinal regions 24. For example, the central longitudinal region 22 may include about 3% by weight or more binder material 40e, while the distal longitudinal regions 24 include less binder material 40e, such as less than about 3% by weight. As a further example, the central longitudinal region 22 may include between about 3% and about 6% by weight binder material 40e, while the distal longitudinal regions 24 may include between about 0% and about 2.5% by weight binder material 40e. Additionally, different types of binder materials 40 may be used in the central longitudinal region 22 versus the distal longitudinal regions 24, as described above.

Yet another approach for forming the absorbent structure 20 having a stabilization profile is to vary the amount of binder activation in the absorbent structure 20. FIG. 5 illustrates the absorbent structure 20 resulting from this approach. A greater amount of energy, such as in the form of heat, can be applied to the central longitudinal region 22 than to either of the distal longitudinal regions 24, thereby allowing for fewer and/or weaker bond points in the distal longitudinal regions 24 of the absorbent structure 20. In this approach, the binder material 40f can be homogeneously mixed with the absorbent material 38 throughout the absorbent structure 20, such that the variation in bond point concentration is attributable to the variation in the amount of binder activation. Alternatively, in addition to varying the amount of binder activation, the amount of binder material 40 may be varied in the CD or transverse direction 34 of the absorbent structure 20, and/or different types of binder material 40 may be used in the central longitudinal region 22 versus the distal longitudinal regions 24, as described above.

As yet another alternative, in addition to, or instead of any of the previously mentioned approaches, the stabilization of the absorbent structure 20 can be modified following activation of the binder material 40. More particularly, some or all of the bond points in the distal longitudinal regions 24 can be broken, such as by pleating the absorbent structure 20 only in selected areas. The unbroken bond points in the central longitudinal region 22 provide greater stabilization than the broken bond points in the distal longitudinal regions 24. Additional stabilization can be provided by embossing the absorbent structure 20, or at least the central longitudinal region 22, with various patterns.

Any combination of approaches for creating the stabilization profile described here can be used without altering the scope of this invention.

Additionally, as illustrated in FIG. 6, the amount of stabilization may also vary in a z-direction of the absorbent structure 20, thereby creating a z-direction stabilization profile. Arrow 42 indicates an axis of the z-direction. In this embodiment, a body side surface 44 of the absorbent structure 20 may have greater stabilization with less stabilization on an outer surface 46 of the absorbent structure 20, as shown in FIG. 6.

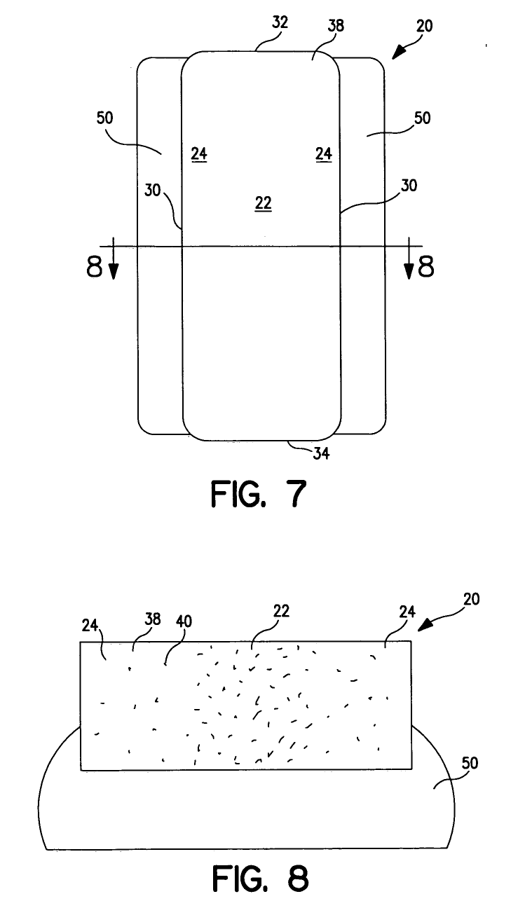

In yet another embodiment, illustrated in FIGS. 7 and 8, the absorbent structure 20 may include a non-stabilized layer 50 of absorbent material adjacent to a surface of the at least partially stabilized absorbent material 38. The non-stabilized layer 50 may have a transverse width about equal to a transverse width of the at least partially stabilized absorbent material 38, or may extend past the distal longitudinal regions 24 of the at least partially stabilized absorbent material 38. The non-stabilized layer 50 provides further capillarity in addition to the capillarity provided in the distal longitudinal regions 24.

The binder material 40 in the absorbent structure 20 may be any suitable binder material 40 that can form bond points under the conditions described herein. For example, the binder material 40 may be binder fibers that can be activated via an external energy source, such as a conventional oven, microwave, infrared, or the like. Alternatively, the binder material 40 can be continuous or discontinuous extruded fibers, including adhesive and/or non-adhesive fibers, non-elastomeric or elastomeric fibers, or a sprayed liquid binder added to the absorbent material 38 during the production or converting process. Examples of suitable spray-on or extruded fibers include meltspray, meltblown, and the like.

Essentially two classes of binder materials 40 can be considered: thermoplastic solid materials (particles or fibers), and liquids (for example, resins or solutions) that can be cured or set by application of heat or other energy sources to provide dry, water-resistant bonds between fibers.

For solid binder materials, any known thermoplastic material can be used as a binder, provided that the material can be fused at a temperature that does not destroy or render unsuitable the absorbent material. A thermoplastic binder upon activation by heat becomes soft but reverts to its normal solid state upon cooling. Representative of such thermoplastic binder materials are polypropylenes, polyethylenes, polycarbonates, polyvinyl chloride, polyesters, polystyrenes, acrylics and the like. The binder material may be hydrophobic or hydrophilic. Hydrophilic fibers can be inherently hydrophilic or can be a synthetic hydrophobic fiber that has been treated with a hydrophilic coating or treatment. Examples of hydrophilic binder fibers are given in U.S. Pat. No. 5,849,000 issued to Anjur et al., incorporated herein by reference in its entirety in a manner consistent with the present document.

The binder material 40 can be monocomponent fibers or bicomponent polymer fibers such as sheath/core fibers or side-by-side bicomponent fiber, having a first component with a lower melting point than the second component, such that upon heating to about the melting point of the first component, the first component can fuse and bond to nearby cellulosic fibers while the second component can maintain the integrity of the binder fiber. Examples include DANAKLON bicomponent fibers of Hercules, Inc. (Wilmington, Del.); or PET (poly(ethylene terephthalate)) core fibers and an activated co-polyethylene sheath, such as CELBOND fibers produced by KoSa Inc. (formerly Trevira Inc. and formerly Hoechst-Celanese), Salisbury, N.C., under the designation T-255 and T-256. Other useful binder fibers include copolyester fibers or materials produced by ES FiberVisions Inc. (Athens, Ga.). In addition to sheath/core fibers, components of a binder fiber having a plurality of polymers may be arranged in an eccentric sheath/core arrangement, a side-by-side arrangement, a pie arrangement or an “islands-in-the-sea” arrangement, or in a blend. Conjugate fibers are taught in U.S. Pat. No. 5,108,820 issued to Kaneko et al., U.S. Pat. No. 5,336,552 issued to Strack et al., and U.S. Pat. No. 5,382,400 issued to Pike et al, each of which is incorporated herein by reference in its entirety in a manner consistent with the present document. For two-component fibers, the polymers may be present in ratios of 75/25, 50/50, 25/75 or any other desired ratios. The fibers may also have shapes such as those described in U.S. Pat. Nos. 5,277,976; 5,069,970; and 5,057,368; which describe fibers with unconventional shapes, each incorporated herein by reference in its entirety in a manner consistent with the present document.

Monocomponent fibers can include, by way of example, polyethylene microfibers marketed as PULPEX fibers by Hercules, Inc. (Wilmington, Del.) or Eastman's KODEL 410 binder fiber. This fiber requires a minimum temperature of about 132 degrees Celsius for good bonding. CoPET B from Eastman Chemical Company is another commercially available binder material with an activation temperature of about 110 degrees Celsius or higher. (This material can also be used as a sheath. For example, a useful bicomponent fiber is a coextruded sheath/core bicomponent with 35% CoPET B and a 65% PET core.)

Fibrous binder material can have a weight-averaged fiber length of about 8 centimeters (cm) or less, specifically from about 0.2 cm to 5 cm, more specifically from 0.3 cm to 3 cm, more specifically still from 0.3 cm to 2 cm, and most specifically from 0.4 cm to 1 cm.

The binder material 40 can also be a microwave-sensitive material having a high dielectric loss constant (for example, from about 1 to 1,000 measured at a frequency of 1 kHz) such that the binder material is heated more than any cellulosic fibers or other fibers or particulates to which the binder material is attached in the absorbent structure when microwave energy is applied. (Cellulose can have a loss factor on the order of about 0.06 at 1 kHz.) Exemplary materials include polyamide or polyvinyl methyl based hot melt adhesives and other thermoplastics known in the art. Polyether block amides, polyvinyl chloride (PVC), and related compounds also have high loss factors. The material can have a loss factor much greater than that of cellulose.

Binder materials can also be applied as liquid resins, slurries, colloidal suspensions, or solutions that become rigid or crosslinked upon application of energy (for example, microwave energy, heat, ultraviolet radiation, and the like). As mentioned, combinations of different types of binder materials may also be used in the absorbent structure 20. For example, the absorbent structure 20 may be formed as a relatively weakly bonded structure using thermoplastic solid materials, and then sprayed, coated, and/or printed with a band of latex or other liquid binding agent in the central longitudinal region 22 to provide enhanced stabilization in the central longitudinal region 22.

Various types of thermosetting binders are known to the art such as amino resins, epoxides, silicones, and the like, as well as elastomeric latex emulsions. Representative thermosetting binder materials which are adapted for application in the form of a liquid dispersion include copolymers of ethylene and acrylic acid, vinyl acetate-ethylene copolymers, acrylonitrile-butadiene copolymers, vinylchloride polymers, vinylidene chloride polymers, curable acrylic latex compositions, and the like.

Water-soluble, non-colloidal, cationic, thermosetting binders suitable for use with cellulosic fibers are disclosed in U.S. Pat. No. 4,617,124 issued to Pall et al., incorporated herein by reference in its entirety in a manner consistent with the present document, where epoxide-based versions are said to be preferred, including both polyamido/polyaminoepichlorohydrin resins and polyamine-epichlorohydrin resins, such as KYMENE 557 and the POLYCUP series of resins manufactured by Hercules Incorporated (Wilmington, Del.). Related materials can be prepared by reacting epichlorohydrin with condensation products of polyalkylene polyamides and ethylene dichloride. Compositions of this type are disclosed in U.S. Pat. No. 3,855,158 and are exemplified by SANTO-RES 31, a product of Monsanto Inc. Another form of this particular type of binder resin is prepared by the reaction of epichlorohydrin with polydiallyl methyl amine to produce an epoxide functional quaternary ammonium resin. Compositions of this kind are disclosed in U.S. Pat. No. 3,700,623 and are exemplified by Resin R4308, a product of Hercules Incorporated. The disclosures of U.S. Pat. Nos. 3,855,158 and 3,700,623 are each incorporated herein by reference in its entirety in a manner consistent with the present document.

The absorbent material 38 may be an airformed composite, for example. The absorbent material 38 can include a matrix of hydrophilic fibers, such as a web of cellulosic fluff, such as wood pulp fluff, or other fiberized pulp fibers, or the fibers can be mixed with particles of a high-absorbency material commonly known as superabsorbent material. One example of a suitable pulp fluff is identified with the trade designation CR1654, available from U.S. Alliance, Childersburg, Ala., U.S.A., and is a bleached, highly absorbent sulfate wood pulp containing primarily soft wood fibers.

The amounts of pulp, binder material, and superabsorbent material may vary considerably depending on the intended application of the absorbent structure 20. More particularly, the absorbent structure may include between about 9% and about 97% by weight pulp, or between about 10% and about 78% by weight pulp, or between about 32% and about 57% by weight pulp. In certain applications the absorbent structure may include between about 9% and about 19% by weight fluff, while in other applications the absorbent structure may include between about 85% and about 95% by weight fluff. The amount of binder material in the absorbent structure 20 is suitably between about 2% and about 20%, or between about 2% and about 15%, or between about 3% and about 8% by weight of the absorbent structure 20. Superabsorbent content in the absorbent structure 20 suitably ranges from about 0% to about 87%, or from about 20% to about 80%, or from about 40% to about 50% by weight of the absorbent structure.

The superabsorbent materials can include particulates, fibers, films, foams, non-ionic superabsorbents, and/or polyacrylate superabsorbents, for example. The superabsorbent materials can be selected from natural, synthetic, and modified natural polymers and materials. The superabsorbent materials can be inorganic materials, such as silica gels, or organic compounds, such as crosslinked polymers. Conventional superabsorbent materials are crosslinked polyelectrolytes. Polyelectrolytes include both anionic and cationic polymers. Anionic polymers contain functional groups such as carboxyl, sulfonate, sulphate, sulfite, phosphate, or a mixture thereof. Examples of anionic polymers include, but are not limited to, salts or partial salts of polyacrylic acid, polyacrylamido methylpropane sulfonic acid, polyvinyl acetic acid, polyvinyl phosphonic acid, polyvinyl sulfonic acid, isobutylene-maleic anhydride copolymer, carboxymethyl cellulose, alginic acid, carrageenan, polyaspartic acid, polyglutamic acid, and copolymers or mixtures thereof. Cationic polymers contain functional groups such as primary, secondary, and tertiary amine, imine, amide, quaternary ammonium, or mixtures thereof. Examples of cationic polymers include, but are not limited to, salts or partial salts of polyvinyl amine, polydiallyl dimethyl ammonium hydroxide, polyacrylamidopropyl trimethyl ammonium hydroxide, polyamino propanol vinyl ether, polyallylamine, chitosan, polylysine, polyglutamine, and copolymers or mixtures thereof. Examples of commercially available superabsorbent materials include SXM 9543 and FAVOR 880, both available from Stockhausen Inc. in Greensboro, N.C., U.S.A., and Dow DRYTECH 2035, available from Dow Chemical Co. in Midland, Mich., U.S.A. These and other superabsorbent materials, including multicomponent superabsorbent material (i.e., superabsorbents with both anionic and cationic polymers) and biodegradable superabsorbents, are suitable for use in the invention.

The absorbent structure 20 can be formed using any suitable absorbent pad-forming process. Examples of suitable processes are described, for example, in U.S. Pat. No. 5,227,107 issued to Dickenson et al., U.S. Pat. No. 6,060,115 issued to Borowski et al., and U.S. Pat. No. 6,470,943 issued to Borowski et al., each of which is incorporated herein by reference in its entirety in a manner consistent with the present document.

A surfactant may also be added to the absorbent structure 20 to increase the hydrophilicity of the absorbent structure 20. Examples of suitable surfactants are commercially available from Uniqema in Wilmington, Del., under the trade designation AHCOVEL, and from Henkel KGAA Corporation in Dusseldorf, Germany, under the trade designation GLUCOPON 220.

The absorbent structure 20 can be incorporated into any suitable absorbent article. Examples of such suitable articles include diapers, training pants, swimwear, absorbent underpants, incontinence products, feminine care products, and the like, as well as medical absorbent articles such as medical absorbent garments, bandages, masks, wound dressings, underpads, wipes, and the like. For ease of explanation, the description hereafter will be in terms of a diaper.

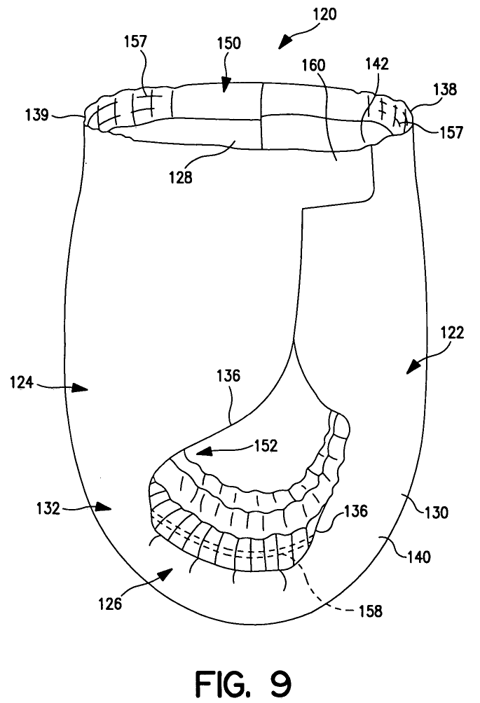

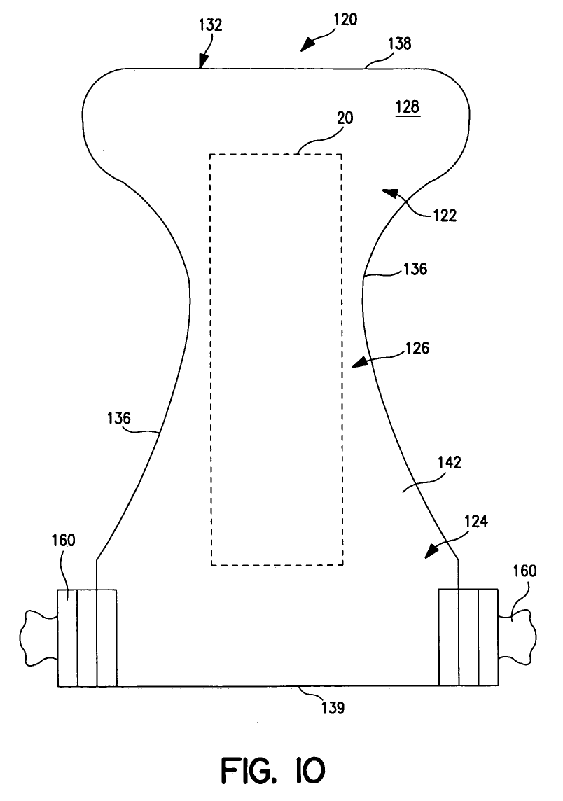

An example of a suitable diaper 120 into which the absorbent structure may be incorporated is illustrated in FIGS. 9 and 10. The diaper 120 includes a chassis 132. The chassis 132 defines a front region 122, a back region 124, a crotch region 126 interconnecting the front region 122 and the back region 124, a body-contacting surface 128 which is configured to contact the wearer, and an outer surface 130 opposite the body-contacting surface 128 which is configured to contact the wearer's clothing. The front region 122 is contiguous with a front waist edge 138, and the back region 124 is contiguous with a back waist edge 139.

The diaper 120 includes an outer cover 140, a body side liner 142 which is connected to the outer cover 140 in a superposed relation, and a pair of side panels 160 attached to the outer cover 140 and/or the body side liner 142. These side panels 160 can be tabs, straps, tearable seams, or similar devices that can be fastened between the front region 122 and the back region 124 by suitable means, including adhesives.

As shown in the diaper 120 in FIG. 9, the front and back regions 122 and 124 together define a three-dimensional pant configuration having a waist opening 150 and a pair of leg openings 152. The waist edges 138 and 139 of the absorbent chassis 132 are configured to encircle the waist of the wearer when worn and provide the waist opening 150 which defines a waist perimeter dimension. Portions of transversely opposed side edges 136 of the chassis 132 in the crotch region 126 generally define the leg openings 152. The front region 122 includes the portion of the diaper 120 which, when worn, is positioned on the front of the wearer while the back region 124 includes the portion of the diaper 120 which, when worn, is positioned on the back of the wearer. The crotch region 126 of the diaper 120 includes the portion of the diaper 120 which, when worn, is positioned between the legs of the wearer and covers the lower torso of the wearer. The side panels 160 of the diaper 120, when worn, are positioned forward from the hips of the wearer.

To enhance the containment of any body exudates discharged from the wearer, the absorbent chassis 132 can include a pair of elasticized containment flaps (not shown) which are configured to provide a barrier to the transverse flow of body exudates. The elasticized containment flaps define an unattached edge which assumes an upright, generally perpendicular configuration in at least the crotch region 126 of the diaper 120 to form a seal against the wearer's body. Suitable constructions and arrangements for the containment flaps are generally well known to those skilled in the art and are described in U.S. Pat. No. 4,704,116 issued Nov. 3, 1987 to Enloe, incorporated herein by reference in its entirety in a manner consistent with the present document.

To further enhance the containment of body exudates, the diaper 120 can include waist elastic members 157 and/or leg elastic members 158, as are known to those skilled in the art (FIG. 9). The waist elastic members 157 can be operatively joined to the outer cover 140 and/or the body side liner 142 along the opposite waist edges 138 and 139, and can extend over part or all of the waist edges. The leg elastic members 158 are desirably operatively joined to the outer cover 140 and/or the body side liner 142 longitudinally along the opposite side edges 136 and positioned in the crotch region 126 of the diaper 120.

The outer cover 140 may include a material that is substantially liquid-impermeable, and can be elastic, stretchable or nonstretchable. The outer cover 140 can be a single layer of liquid-impermeable material, or may include a multi-layered laminate structure in which at least one of the layers is liquid-impermeable. For instance, the outer cover 140 can include a liquid-permeable outer layer and a liquid-impermeable inner layer that are suitably joined together by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid-permeable outer layer can be any suitable material such as one that provides a generally cloth-like texture. One example of such a material is a 20 gsm (grams per square meter) spunbond polypropylene nonwoven web. The outer layer may also be made of those materials of which liquid-permeable body side liner 142 is made. While it is not a necessity for the outer layer to be liquid-permeable, it is desired that it provides a relatively cloth-like texture to the wearer.

The inner layer of the outer cover 140 can be both liquid and vapor-impermeable, or can be liquid-impermeable and vapor-permeable. The inner layer may be manufactured from a thin plastic film, although other flexible liquid-impermeable materials may also be used. The inner layer, or the liquid-impermeable outer cover 140 when a single layer, prevents waste material from wetting articles, such as bedsheets and clothing, as well as the wearer and care giver. A suitable liquid-impermeable film for use as a liquid-impermeable inner layer, or a single layer liquid-impermeable outer cover 140, is a 0.02 millimeter polyethylene film commercially available from Huntsman Packaging of Newport News, Va., U.S.A. If the outer cover 140 is a single layer of material, it can be embossed and/or matte finished to provide a more cloth-like appearance. As earlier mentioned, the liquid-impermeable material can permit vapors to escape from the interior of the disposable absorbent article, while still preventing liquids from passing through the outer cover 140. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

The liquid-permeable body side liner 142 is illustrated as overlying the outer cover 140 and absorbent structure 20, and may but need not have the same dimensions as the outer cover 140. The body side liner 142 is desirably compliant, soft feeling, and non-irritating to the wearer's skin. Further, the body side liner 142 can be less hydrophilic than the absorbent structure 20, to present a relatively dry surface to the wearer and permit liquid to readily penetrate through its thickness.

The body side liner 142 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the body side liner 142. For example, the body side liner can be composed of a meltblown or spunbonded web of polyolefin fibers. The body side liner can also be a bonded-carded web composed of natural and/or synthetic fibers. The body side liner can be composed of a substantially hydrophobic material, and the hydrophobic material can, optionally, be treated with a surfactant or otherwise processed to impart a desired level of wettability and hydrophilicity.

The absorbent structure 20 can be positioned or located in a relatively flat or unfolded configuration between the outer cover 140 and the body side liner 142, which components can be joined together by any suitable means, such as adhesives, as are well known in the art. FIG. 10 illustrates the diaper 120 with the absorbent structure 20 incorporated therein.

The chassis 132 can also incorporate other materials that are designed primarily to receive, temporarily store, and/or transport liquid along the mutually facing surface with the absorbent structure, thereby maximizing the overall absorbent capacity of the absorbent structure 20, if desired. One suitable material is referred to as a surge layer (not shown) and includes a material having a basis weight of about 50 to about 120 grams per square meter (gsm), and including a through-air-bonded-carded web of a homogenous blend of 60 percent 3 denier type T-256 bicomponent fiber including a polyester core/polyethylene sheath and 40 percent 6 denier type T-295 polyester fiber, both commercially available from Kosa Corporation of Salisbury, N.C., U.S.A. Another example of a suitable surge layer may include a material made of 6 denier polyethylene terephthalate (PET) and 6 denier bicomponent binder fiber, having a basis weight of about 50 to about 120 gsm.

The various components of the diaper 120 can be integrally assembled together employing various types of suitable attachment means, such as adhesive, ultrasonic and thermal bonds or combinations thereof.

As described herein, the absorbent structure 20 of the invention provides integrity and permeability to the target zone 36 of the absorbent structure 20 with reduced overall stiffness compared to a fully stabilized absorbent structure.

### EXAMPLE

This example verified the stiffness reduction gained by varying binding in a stabilized absorbent structure.

Two types of samples were made for testing, designated Type A and Type B. All samples had a basis weight of 500 grams per square meter (gsm) and were cut to 2 inches (5 cm) wide by 12 inches (30 cm) long. Type A samples contained 50% SXM 9543 superabsorbent available from Stockhausen, Inc. of Greensboro, N.C.; 45% NB-416 pulp available from Weyerhaeuser of Federal Way, Wash.; and 5% T-255 binder fiber available from KoSa of Houston, Tex.; throughout the samples. Type B samples contained the same components in the middle 1 inch (3 cm) width of each sample. The outer 0.5 inch (1 cm) width on each side of Type B samples contained 50% SXM 9543 superabsorbent, 50% NB-416 pulp, and no binder fiber. All samples were heated in a conventional hot air oven at 170 degrees Celsius for 5 minutes to activate the binder.

After oven bonding, samples were tested for cylindrical compression using the test method described below. The two longitudinal ends of each sample were stapled together forming cylindrical rings having a height of 2 inches (5 cm). The rings were then placed under load and the load required to crush the ring to less than 50% of the original height was measured. Results of this stiffness testing are presented in Table 1.

TABLE 1
Stiffness Data

| | | | |
|---|---|---|---|
| Sample | Binder Content in | Binder Content Along | Peak Load (g) to |
| Type | Middle | Outer Edges | Compress Samples |

| | | | |
|---|---|---|---|
| A | 5% | 5% | 222 |
| B | 5% | No binder | 161 |

The above data illustrates that the measured stiffness of the entire absorbent structure can be decreased by decreasing the binder material content on the outer edges.

Cylindrical Compression Test Method

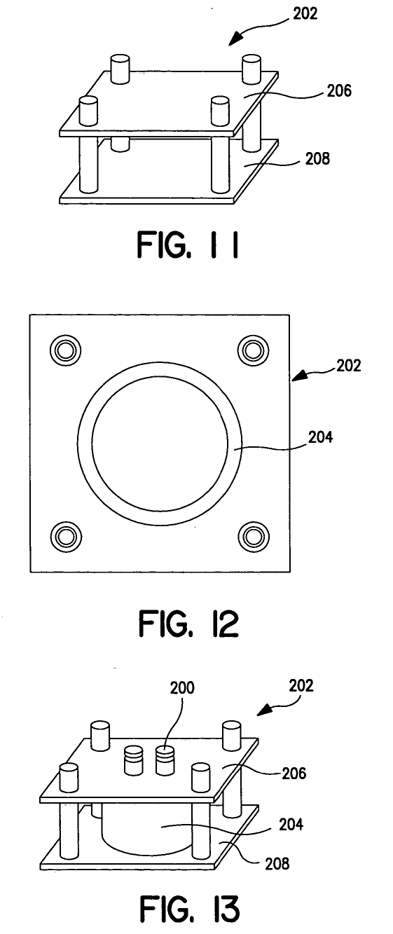

Equipment required for carrying out the test includes: a paper cutter, a stapler, several 100-gram weights 200, and a testing apparatus 202 as illustrated in FIGS. 11-13. The testing apparatus 202 includes a top plate 206 positioned above a bottom plate 208. Both the top plate 206 and the bottom plate 208 are constructed of transparent LEXAN® polycarbonate material, available from General Electric Company. The top plate 206 includes four holes into which four posts extending from the bottom plate 208 are inserted to maintain the top plate 206 in alignment over the bottom plate 208.

1. Cut a 2-inch by 12-inch sample 204 of the test material with a paper cutter. Make sure the 12-inch dimension is in the machine direction.

2. Wrap the sample 204 into a cylinder and staple the ends together with two staples, one near the top and one near the bottom of the cylinder.

3. Place the sample between the top plate 206 and the bottom plate 208.

4. Place one 100-gram weight at a time, cumulatively, on the top plate 206, as shown in FIG. 13, until the sample fails.

5. Record the total load placed on the sample.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

1. An absorbent structure, comprising:
a central longitudinal region;
two distal longitudinal regions located along opposite sides of the central longitudinal region; and
an absorbent material at least partially stabilized with a binder material and located in the central longitudinal region and in each of the two distal longitudinal regions, wherein the absorbent material is more stabilized in the central longitudinal region than in either of the two distal longitudinal regions.

2. The absorbent structure of claim 1, wherein the central longitudinal region includes a higher concentration of bond points than in either of the two distal longitudinal regions.

3. The absorbent structure of claim 1, wherein the absorbent structure has an essentially uniform bond point concentration across the central longitudinal region and the two distal longitudinal regions.

4. The absorbent structure of claim 1, wherein the central longitudinal region comprises a first binder material, and each of the two distal longitudinal regions comprises a second binder material having a higher melting point than the first binder material.

5. The absorbent structure of claim 1, wherein the central longitudinal region comprises a greater binder-material-to-absorbent-material ratio than a binder-material-to-absorbent-material ratio in each of the two distal longitudinal regions.

6. The absorbent structure of claim 5, wherein the binder-material-to-absorbent-material ratio in the central longitudinal region is about 3% by weight or greater.

7. The absorbent structure of claim 5, wherein the binder-material-to-absorbent-material ratio in each of the two distal longitudinal regions is less than about 3% by weight.

8. The absorbent structure of claim 1, wherein the amount of stabilization in the absorbent material varies in a z-direction through the absorbent structure.

9. The absorbent structure of claim 1, wherein the concentration by weight of absorbent material is essentially the same in the central region as in each of the two distal longitudinal regions.

10. The absorbent structure of claim 1, wherein the absorbent material and the binder material are homogeneously mixed in the central longitudinal region and in the two distal longitudinal regions.

11. The absorbent structure of claim 1, wherein the binder material comprises at least one of the group consisting of binder fibers, continuous extruded fibers, discontinuous extruded fibers, adhesive fibers, non-adhesive fibers, non-elastomeric fibers, elastomeric fibers, sprayed liquid binder, and combinations thereof.

12. The absorbent structure of claim 1, wherein the binder material comprises bicomponent staple fibers.

13. The absorbent structure of claim 1, wherein the absorbent material comprises an airformed composite.

14. The absorbent structure of claim 1, wherein the absorbent material comprises fiberized pulp fibers.

15. The absorbent structure of claim 1, wherein the absorbent material comprises superabsorbent material.

16. The absorbent structure of claim 1, further comprising a non-stabilized layer of absorbent material in contact with a surface of the at least partially stabilized absorbent material.

17. The absorbent structure of claim 1, having a gradual stabilization profile between the central longitudinal region and the two distal longitudinal regions.

18. An absorbent article, comprising:
an outer cover;
a body side liner; and
an absorbent structure positioned between the outer cover and the body side liner, wherein the absorbent structure comprises a central longitudinal region, two distal longitudinal regions located along opposite sides of the central longitudinal region, and an absorbent material located in the central longitudinal region and in each of the two distal longitudinal regions, with the absorbent material more stabilized in the central longitudinal region than in either of the two distal longitudinal regions.

19. The absorbent article of claim 18, wherein the central longitudinal region includes a higher concentration of bond points than in either of the two distal longitudinal regions.

20. The absorbent article of claim 18, wherein the absorbent structure has an essentially uniform bond point concentration across the central longitudinal region and the two distal longitudinal regions.

21. The absorbent article of claim 18, wherein the central longitudinal region comprises a first binder material, and each of the two distal longitudinal regions comprises a second binder material having a higher melting point than the first binder material.

22. The absorbent article of claim 18, wherein the central longitudinal region comprises a greater binder-material-to-absorbent-material ratio than a binder-material-to-absorbent-material ratio in each of the two distal longitudinal regions.

23. The absorbent article of claim 18, wherein the absorbent structure further comprises a non-stabilized layer of absorbent material in contact with a surface of the at least partially stabilized absorbent material.

24. The absorbent article of claim 18, wherein the absorbent article is selected from the group consisting of diapers, training pants, swim wear, absorbent underpants, adult incontinence products, feminine care products, medical absorbent garments, bandages, masks, wound dressings, underpads, and wipes.

25. A method of forming an absorbent structure, comprising the steps of:
forming an absorbent material into an absorbent structure; and
at least partially stabilizing the absorbent material with a binder material such that the absorbent material is more stabilized in a central longitudinal region than in either of two distal longitudinal regions located along opposite sides of the central longitudinal region.

26. The method of claim 25, further comprising the step of heating the absorbent structure to activate the binder material.

27. The method of claim 25, comprising the step of activating the binder material in the central longitudinal region to a greater extent than the binder material in the two distal longitudinal regions.

28. The method of claim 25, wherein the central longitudinal region comprises a first binder material, and each of the two distal longitudinal regions comprises a second binder material having a higher melting point than the first binder material.

29. The method of claim 25, comprising stabilizing the absorbent structure by applying a greater concentration of binder material to the central longitudinal region of the absorbent structure than to either of the two distal longitudinal regions.

30. The method of claim 25, further comprising the step of breaking a plurality of bond points in each of the two distal longitudinal regions.

31. The method of claim 25, further comprising applying a non-stabilized layer of absorbent material to the at least partially stabilized absorbent material.
